(19) United States
(12) Patent Application Publication
(10) Pub. No.: US 2009/0306098 A1
(43) Pub. Date: Dec. 10, 2009

(54) COMBINATION OF ROSCOVITINE AND A HDCA INHIBITOR TO TREAT PROLIFERATIVE DISEASES

(75) Inventors: Simon Green, Perth and Kinross (GB); Sheelagh Frame, Angus (GB); Ian Fleming, Angus (GB)

Correspondence Address: LAHIVE & COCKFIELD, LLP;FLOOR 30, SUITE 3000, ONE POST OFFICE SQUARE, BOSTON, MA, 02109, US

(73) Assignee: Cyclacel Limited, London (GB)

(21) Appl. No.: 12/093,418

(22) PCT Filed: Nov. 13, 2006
(86) PCT No.: PCT/GB06/04226

§ 371 Date: Oct. 7, 2008

(30) Foreign Application Priority Data
Nov. 11, 2005 (GB) 0523040.4

(51) Int. Cl. A61K 31/52; A61P 35/00

(52) U.S. Cl. 514/263.4

(57) ABSTRACT

A first aspect of the invention relates to a combination comprising roscovitine, or a pharmaceutically acceptable salt thereof, and a HDAC inhibitor selected from sodium butyrate, or a prodrug thereof, suberoylanilide hydroxamic acid (SAHA), sodium valproate and trichostatin A (TSA). A second aspect of the invention relates to a pharmaceutical product comprising roscovitine, or a pharmaceutically acceptable salt thereof, and a HDAC inhibitor selected from sodium butyrate, or a prodrug thereof, suberoylanilide hydroxamic acid (SAHA), sodium valproate and trichostatin A (TSA) as a combined preparation for simultaneous, sequential or separate use in therapy. A third aspect of the invention relates to a method for treating a proliferative disorder, said method comprising simultaneously, sequentially or separately administering roscovitine, or a pharmaceutically acceptable salt thereof, and a HDAC inhibitor selected from sodium butyrate, or a prodrug thereof, suberoylanilide hydroxamic acid (SAHA), sodium valproate and trichostatin A (TSA) to a subject.

The present invention relates to a pharmaceutical combination suitable for the treatment of proliferative disorders. In particular, the present invention relates to combinations for the treatment of cancer, preferably non-small cell lung cancer (NSCLC).

### BACKGROUND TO THE INVENTION

Cyclin-dependent kinases (CDKs) are serine/threonine kinases that play a crucial regulatory role in the cell cycle. CDKs regulate cell cycle progression by phosphorylation of various proteins involved in DNA replication and cell division, including transcription factors and tumour suppressor proteins (Senderowicz, A M. Small-molecule cyclin-dependent kinase modulators, Oncogene, 2003; 22: 6609-6620). Certain CDKs also play a role in the regulation of RNA synthesis by their involvement in the phosphorylation of the carboxy terminal domain (CTD) of the largest subunit of RNA polymerase II (pol II). It is not surprising, therefore, that CDKs have become attractive therapeutic targets. Consequently, many new pharmacological agents capable of interfering with the activity of CDKs by competing for their ATP binding site are currently being tested in clinical trials (Fischer P M and Gianella-Borradori A, CDK inhibitors in clinical development for the treatment of cancer, Expert Opin Investig Drugs. 2003; 12: 955-970).

The prior art has described several compounds that are capable of regulating the cell cycle by virtue of inhibiting cyclin dependent kinases. These compounds include butyrolactone, flavopiridol and 2-(2-hydroxyethylamino)-6-benzylamino-9-methylpurine (olomoucine). Olomoucine and related compounds have been shown to be inhibitors of cdc2. Cdc2 (also known as cdk1) is a catalytic sub-unit of a family of cyclin dependent kinases that are involved in cell cycle regulation.

These kinases comprise at least two sub-units, namely a catalytic sub-unit (of which cdc2 is the prototype) and a regulatory sub-unit (cyclin). The cdks are regulated by transitory association with a member of the cyclin family: cyclin A (cdc2, CDK2), cyclin B1-B3 (cdc2), cyclin C(CDK8), cyclin D1-D3 (CDK2-CDK4-CDK5-CDK6), cyclin E (CDK2), cyclin H(CDK7).

Each of these complexes is involved in a phase of the cellular cycle. CDK activity is regulated by post-translatory modification, by transitory associations with other proteins and by modifications of their intra-cellular localization. The CDK regulators comprise activators (cyclins, CDK7/cyclin H, cdc25 phosphateses), the p9.sup.CKS and p15.sup.CDK-BP sub-units, and the inhibiting proteins (p16.sup.INK4A, p15.sup.INK4B, p21.sup.Cipl, p18, p27.sup.Kipl).

There is now considerable support in the literature for the hypothesis that CDKs and their regulatory proteins play a significant role in the development of human tumors. Thus, in numerous tumors a temporal abnormal expression of cyclin-dependent kinases, and a major de-regulation of protein inhibitors (mutations, deletions) has been observed.

Roscovitine has been demonstrated to be a potent inhibitor of cyclin dependent kinase enzymes, particularly CDK. CDK inhibitors are understood to block passage of cells from the G1/S and the G2/M phase of the cell cycle. The pure R-enantiomer of roscovitine, seliciclib (R-Roscovitine; CYC202) has recently emerged as a potent inducer of apoptosis in a variety of tumour cells (McClue S J, Blake D, Clarke R, et al, In vitro and in vivo antitumor properties of the cyclin dependent kinase inhibitor CYC202 (R-Roscovitine), Int J. Cancer. 2002; 102: 463-468) and is already in clinical trials to treat breast cancer and non-small cell lung cancer (Fischer PM and Gianella-Borradori A, CDK inhibitors in clinical development for the treatment of cancer, Expert Opin Investig Drugs, 2003; 12: 955-970). Roscovitine has also been shown to be an inhibitor of retinoblastoma phosphorylation and therefore implicated as acting more potently on Rb positive tumors.

It well established in the art that active pharmaceutical agents can often be administered in combination in order to optimise the treatment regime. For example, the use of a CDK inhibitor in combination with a second chemotherapeutic agent is described in WO 03/077999, WO 03/082337, WO 2004/041262, WO 2004/041267, WO 2004/041268, WO 2004/041308, WO 2004/110455 and WO 2005/053699 (all to Cyclacel Limited).

The present invention seeks to provide a new combination of known pharmaceutical agents that is particularly suitable for the treatment of proliferative disorders, especially cancer. More specifically, a preferred aspect of the invention centres on combinations useful in the treatment of non-small cell lung cancer (NSCLC).

### STATEMENT OF THE INVENTION

A first aspect of the present invention relates to a combination comprising roscovitine, or a pharmaceutically acceptable salt thereof, and a histone deacetylase (HDAC) inhibitor selected from sodium butyrate, or a prodrug thereof, suberoylanilide hydroxamic acid (SAHA), sodium valproate and trichostatin A (TSA).

Although roscovitine and the above-mentioned HDAC inhibitors are well established in the art as individual therapeutic agents, there has been no suggestion that the specific combinations claimed in the present invention would be effective in the treatment of cancer. Moreover, there has been no suggestion in the art that the specifically claimed combinations would be useful in the treatment of NSCLC, which is known to be particularly difficult to treat.

A second aspect relates to a pharmaceutical composition comprising a combination according to the invention and a pharmaceutically acceptable carrier, diluent or excipient.

A third aspect relates to the use of a combination according to the invention in the preparation of a medicament for treating a proliferative disorder.

A fourth aspect relates to a pharmaceutical product comprising roscovitine, or a pharmaceutically acceptable salt thereof, and a HDAC inhibitor selected from sodium butyrate, or a prodrug thereof, suberoylanilide hydroxamic acid (SAHA), sodium valproate and trichostatin A (TSA), as a combined preparation for simultaneous, sequential or separate use in therapy.

A fifth aspect relates to a method of treating a proliferative disorder, said method comprising simultaneously, sequentially or separately administering roscovitine, or a pharmaceutically acceptable salt thereof, and a HDAC inhibitor selected from sodium butyrate, or a prodrug thereof, suberoylanilide hydroxamic acid (SAHA), sodium valproate and trichostatin A (TSA).

A sixth aspect relates to the use of roscovitine, or a pharmaceutically acceptable salt thereof, in the preparation of a medicament for the treatment of a proliferative disorder, wherein said treatment comprises simultaneously, sequentially or separately administering a HDAC inhibitor selected from sodium butyrate, or a prodrug thereof, suberoylanilide hydroxamic acid (SAHA), sodium valproate and trichostatin A (TSA).

A seventh aspect relates to the use of a HDAC inhibitor selected from sodium butyrate, or a prodrug thereof, suberoylanilide hydroxamic acid (SAHA), sodium valproate and trichostatin A (TSA) in the preparation of a medicament for the treatment of a proliferative disorder, wherein said treatment comprises simultaneously, sequentially or separately administering roscovitine, or a pharmaceutically acceptable salt thereof.

An eighth aspect relates to the use of (i) roscovitine, or a pharmaceutically acceptable salt thereof, and (ii) a HDAC inhibitor selected from sodium butyrate, or a prodrug thereof, suberoylanilide hydroxamic acid (SAHA), sodium valproate and trichostatin A (TSA), in the preparation of a medicament for treating a proliferative disorder.

A ninth aspect relates to the use of a HDAC inhibitor selected from sodium butyrate, or a prodrug thereof, suberoylanilide hydroxamic acid (SAHA), sodium valproate and trichostatin A (TSA), in the preparation of a medicament for treating a proliferative disorder, wherein said medicament is for use in combination therapy with roscovitine, or a pharmaceutically acceptable salt thereof.

A tenth aspect relates to the use of a HDAC inhibitor selected from sodium butyrate, or a prodrug thereof, suberoylanilide hydroxamic acid (SAHA), sodium valproate and trichostatin A (TSA), in the preparation of a medicament for treating a proliferative disorder, wherein said medicament is for use in pretreatment therapy with roscovitine, or a pharmaceutically acceptable salt thereof.

An eleventh aspect relates to the use of roscovitine, or a pharmaceutically acceptable salt thereof, in the preparation of a medicament for treating a proliferative disorder, wherein said medicament is for use in combination therapy with a HDAC inhibitor selected from sodium butyrate, or a prodrug thereof, suberoylanilide hydroxamic acid (SAHA), sodium valproate and trichostatin A (TSA).

A twelfth aspect relates to the use of roscovitine, or a pharmaceutically acceptable salt thereof, in the preparation of a medicament for treating a proliferative disorder, wherein said medicament is for use in pretreatment therapy with a HDAC inhibitor selected from sodium butyrate, or a prodrug thereof, suberoylanilide hydroxamic acid (SAHA), sodium valproate and trichostatin A (TSA).

A thirteenth aspect relates to the use of roscovitine, or a pharmaceutically acceptable salt thereof, and a HDAC inhibitor, in the preparation of a medicament for the treatment of non small cell lung cancer (NSCLC).

### DETAILED DESCRIPTION

The preferred embodiments set out below are applicable to all the above-mentioned aspects of the invention.

Roscovitine or 2-[(1-ethyl-2-hydroxyethyl)amino]-6-benzylamine-9-isopropylpurine, is also described as 2-(1-D,L-hydroxymethylpropylamino)-6-benzylamine-9-isopropyl-purine. As used herein, the term “roscovitine” encompasses the resolved R and S enantiomers, mixtures thereof, and the racemate thereof.

As used herein, the term “seliciclib” refers to the R enantiomer of roscovitine, namely, 2-(1-R-hydroxymethylpropylamino)-6-benzylamino-9-isopropylpurine, the structure of which is shown below.

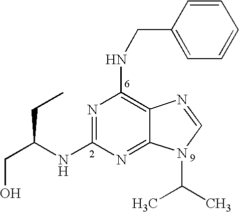

For all embodiments of the invention, preferably roscovitine is in the form of the R enantiomer, namely 2-(1-R-hydroxymethylpropylamino)-6-benzylamino-9-isopropyl-purine, hereinafter referred to as “seliciclib” or “CYC202” or “R-roscovitine”.

The in vitro activity of roscovitine is as follows:

| Kinase | IC₅₀ (μM) |
|---|---|
| Cdk1/cyclin B | 2.7 |
| Cdk2/cyclin A | 0.7 |
| Cdk2/cyclin E | 0.1 |
| Cdk7/cyclin H | 0.5 |
| Cdk9/cyclin T1 | 0.8 |
| Cdk4/cyclin D1 | 14.2 |
| PKA | >50 |
| PKC | >50 |

As mentioned above, the presently claimed combinations comprise roscovitine and a histone deacetylase (HDAC) inhibitor.

Histones are small positively charged proteins that are rich in basic amino acids (positively charged at physiological pH). There are five main types of histones namely, H1, H2A, H2B, H3, and H4 which exhibit a high degree of structural similarity. Histones are not found in eubacteria (e.g., E. coli), although the DNA of these bacteria is associated with other proteins that presumably function like histones to package the DNA within the bacterial cell. Archaeabacteria, however, do contain histones that package their DNAs in structures similar to eukaryotic chromatin (G. M. Cooper, “The Cell—A Molecular Approach”, 2^{nd} Edition, Chapter II).

The majority of histones are synthesized during the S phase of the cell cycle, and newly synthesized histones quickly enter the nucleus to become associated with DNA. Within minutes of its synthesis, new DNA becomes associated with histones in nucleosomal structures.

The amino-terminal tail domains of histones may be enzymatically modified by post-translational addition of methyl (to lysine and arginine groups), acetyl (to lysine groups), or phosphate groups (to serine groups) (Spencer et al, Gene, 1999, 240(1), 1). This results in a reduction of the net positive charge of the histone which, consequently, may weaken the binding of the histone to DNA.

Studies of histone deacetylators (HDACs), as well as the compounds which inhibit HDACs, have elucidated the mechanisms through which some disease states act. For example, in the search for novel anti-malarial compounds, the naturally occurring apicidin was shown to inhibit the in vitro growth of P. falciparum by hyperacetylating histones (K. T. Andrews et al, Int. J. Parasitol., 2000, 30(6), 761).

HDACs, therefore, are believed to be associated with a number of different diseases which include proliferative disorders such as leukemia (Lin et al, Nature, 1998, 391, 811), melanomas/squamous cell carcinomas (Gillenwater et al, Int. J. Cancer, 1998, 75217; Saunders et al, Cancer Res., 1999, 59, 399), breast cancer, prostrate cancer, bladder cancer (Gelmetti et al, Mol. Cell. Biol., 1998, 18, 7185; Wang et al, PNAS, 1998, 951, 10860) and colon cancer (C. A. Hassig, et al, 1997, Chem. Biol., 4, 783; S. Y. Archer et al, PNAS, 1998, 95(12), 6791).

US 2005/0004007 discloses a method for promoting apoptosis in cancer cells which involves administering a cyclin dependent kinase inhibitor and an agent which induces cellular differentiation. Several categories of agent which induce cellular differention are given namely, histone deacetylase inhibitors, protein kinase C, retinoids and vitamin D3. However, combinations comprising roscovitine and a HDAC inhibitor are not specifically disclosed, nor is the use of this combination in the treatment of solid tumours, such as NSCLC. On the contrary, the exemplification of US 2005/0004007 is limited to combinations of flavopiridol with selected HDAC inhibitors tested on leukemia cell lines.

Accordingly, to date, there has been no disclosure of the specific combinations claimed in the present application, let alone any suggestion that they would be therapeutically useful in the treatment of lung cancers such as NSCLC.

In one preferred embodiment of the invention, the HDAC inhibitor is sodium butyrate.

Sodium butyrate is formed on the fermentation of dietary fibres in the lumen of the large intestine (G. J. Kelloff et al, Cancer Chemoprevention: Volume 1, page 665). It has been found to increase the expression of exon 7-containing SMN protein from the SMN2 gene in spinal muscular atrophy lymphoid cell lines. It was proposed that sodium butyrate worked by acetylating nucleosomal DNA and other factors that control alternating splicing of exon 7 of the SMN42 gene (J.-G. Chang et al, PNAS, 2001, 98(17), 9809).

Sodium butyrate has also been shown to induce differentiation in cultured erythroleukemic cells (A. Leder et al, Cell, 1975, 5(3), 319) and PC12 pheochromocytoma cells (J. C. Byrd et al, Brain Res., 1987, 428(1), 151), along with increasing the expression of fetal hemoglobin genes in humans and other animals (S. P. Perrine et al, Adv. Exp. Med. Biol., 1989, 271, 177 and S. P. Perrine et al, PNAS, 1988, 85(22), 8540).

In another preferred embodiment, the HDAC inhibitor is a prodrug of sodium butyrate.

In a particularly preferred embodiment, the prodrug is pivaloyloxymethyl butyrate. Pivaloyloxymethyl butyrate (Pivanex®) is an acyloxyalkyl ester prodrug of butyric acid and has been shown to induce the instrinsic pathway of apoptosis in leukemia and neuroblastoma cells (S. Mei et al, International Journal of Oncology, 2004, 25, 1509).

In another preferred embodiment of the invention, the HDAC inhibitor is trichostatin A (TSA).

The antifungal antibiotic trichostatin was first isolated from the metabolites of strains of Streptomyces hygroscopicus (N. Tsuji et al, J. Antibiot., 1976, 29, 1). Trichostatin A (TSA) is a specific and reversible inhibitor of HDAC. At nanomolar concentrations, TSA causes a marked accumulation of highly acetylated histones in vivo and strongly inhibits the activity of the partially purified histone deacetylase in vitro (M. Yoshida et al, J. Biol. Chem., 1990, 265(28), 17174). In human Jerkat T cells, TSA arrests cell cycle progression in G1 and inhibits the activity of the HD1 deacetylase with an IC₅₀ of 70 nM (Y. Hoshikawa et al, Exp. Cell Res., 1994, 214, 189).

TSA can also concomitantly modify the expression of genes. Mishra et al demonstrated that TSA significantly downregulated CD154 and IL-10 and up-regulated IFN-γ gene expression in systemic lupus erythematosis (SLE) T cells. SLE is an autoimmune disease characterised by dysregulated production of antibodies which leads to irreversible, immune complex-mediated end-organ failure (N. Mishra et al, PNAS, 2001, 98(5), 2628).

In another preferred embodiment of the invention, the HDAC inhibitor is suberoylanilide hyroxamic acid (SAHA).

Suberoylanilide hyroxamic acid (SAHA) is a synthetic derivative of TSA which inhibits HDAC activity at micromolar concentrations. SAHA is currently undergoing Phase II clinical trials in the US for its use in the treatment of relapsed or refractory advanced Hodgkin's lymphoma.

The antiproliferative effect of SAHA is well documented. WO 2005/097747 (Aton Pharma) discloses the use of prodrugs of hydroxamic based HDAC inhibitors, such as SAHA, in the treatment of neoplasms, thioredoxin (TRX)-mediated diseases and in the prevention and/or treatment of CNS diseases.

US 2004/4127525 (Bacopoulos et al) discloses the use of SAHA in the treatment of lymphomas, such as diffuse large B-cell lymphoma. WO 2005/039498 and WO 2005/018578 (both to Aton Pharma) disclose further methods for the treatment of neoplasms, wherein WO2005/039498 relates to leukemia and WO 2005/018578 relates to mesothelioma or lymphoma.

In one preferred embodiment of the invention, the HDAC inhibitor is sodium valproate (otherwise known as sodium 2-propylpentanoate). Sodium valproate is the sodium salt of valproic acid and is a NICE-approved anticonvulsant drug used in the treatment of epilepsy. More recently, studies have investigated the use of sodium valproate for the treatment of advanced solid tumour malignancies and cancer-related neuropathic pain. Combination studies involving valproic acid and UCN-01 have also been undertaken. In this regard, although valproic acid itself has only a weak anticancer effect, studies have shown that it becomes highly effective against cancer cells when used in combination.

Another aspect of the present invention relates to a pharmaceutical composition comprising roscovitine, or a pharmaceutically acceptable salt thereof, and a HDAC inhibitor selected from sodium butyrate, or a prodrug thereof, suberoylanilide hydroxamic acid (SAHA), sodium valproate and trichostatin A (TSA).

Another aspect relates to a pharmaceutical product comprising the combination of the present invention for use in the treatment of a proliferative disorder, wherein the disorder is preferably cancer, and more preferably, NSCLC.

A further aspect of the present invention relates to a pharmaceutical product comprising roscovitine, or a pharmaceutically acceptable salt thereof, and a HDAC inhibitor selected from sodium butyrate, or a prodrug thereof, suberoylanilide hydroxamic acid (SAHA), sodium valproate and trichostatin A (TSA), as a combined preparation for simultaneous, sequential or separate use in therapy.

Yet another aspect relates to a method of treating a proliferative disorder, said method comprising simultaneously, sequentially or separately administering a combination of the present invention.

As used herein, “simultaneously” is used to mean that the two agents are administered concurrently, whereas the term “in combination” is used to mean that they are administered, if not simultaneously, then “sequentially” with a time frame that they are able to act therapeutically within the same time frame. Thus, administration “sequentially” may permit one agent to be administered within 5 minutes, 10 minutes or a matter of hours after the other provided that they are both concurrently present in therapeutic amounts. The time delay between administration of the components will vary depending on the exact nature of the components, the interaction therebetween and their respective half-lives.

In contrast to “in combination” or “sequentially”, “separately” is used herein to mean that the gap between administering one agent and the other is significant i.e. the first administered agent may no longer be present in the bloodstream in a therapeutically effective amount when the second agent is administered.

In one preferred embodiment, the HDAC inhibitor is administered sequentially or separately prior to roscovitine, or a pharmaceutically acceptable salt thereof.

In another preferred embodiment, the roscovitine, or pharmaceutically acceptable salt thereof, is administered sequentially or separately prior to the HDAC inhibitor.

In another preferred embodiment, the HDAC inhibitor and roscovitine, or pharmaceutically acceptable salt thereof, are administered concurrently.

Where the HDAC inhibitor is sodium butyrate, or a prodrug thereof, or TSA, the HDAC inhibitor and roscovitine may be administered simultaneously, or separately or sequentially, irrespective of the order of administration.

Where the HDAC inhibitor is SAHA, preferably the SAHA is administered prior to the roscovitine, i.e. preferably, the subject is pretreated with SAHA.

Where the HDAC inhibitor is sodium valproate, preferably the roscovitine and sodium valproate are administered separately or sequentially, irrespective of the order of administration.

In a preferred embodiment, roscovitine, or a pharmaceutically acceptable salt thereof, and the HDAC inhibitor are each administered in a therapeutically effective amount with respect to the individual components.

In another preferred embodiment, roscovitine, or pharmaceutically acceptable salt thereof, and the HDAC inhibitor are each administered in a sub-therapeutic amount with respect to the individual components.

The term “sub-therapeutic amount” means an amount that is lower than that typically required to produce a therapeutic effect with respect to treatment with roscovitine alone or the HDAC inhibitor alone.

A further aspect relates to the use of the combination of the present invention in the preparation of a medicament for treating a proliferative disorder.

As used herein the phrase “preparation of a medicament” includes the use of one or more of the above described components directly as the medicament or in any stage of the manufacture of such a medicament.

Another aspect relates to the use of roscovitine, or a pharmaceutically acceptable salt thereof, in the preparation of a medicament for the treatment of a proliferative disorder, wherein said treatment comprises simultaneously, sequentially or separately administering a HDAC inhibitor selected from sodium butyrate, or a prodrug thereof, suberoylanilide hydroxamic acid (SAHA), sodium valproate and trichostatin A (TSA) to a subject.

Yet another aspect relates to the use of a HDAC inhibitor selected from sodium butyrate, or a prodrug thereof, suberoylanilide hydroxamic acid (SAHA), sodium valproate and trichostatin A (TSA), in the preparation of a medicament for the treatment of a proliferative disorder, wherein said medicament is for use in combination therapy with roscovitine, or a pharmaceutically acceptable salt thereof. Alternatively, the therapy can be pretreatment therapy.

A further aspect relates to the use of roscovitine, or a pharmaceutically acceptable salt thereof, in the preparation of a medicament for the treatment of a proliferative disorder, wherein said medicament is for use in combination therapy with a HDAC inhibitor selected from sodium butyrate, or a prodrug thereof, suberoylanilide hydroxamic acid (SAHA), sodium valproate and trichostatin A (TSA). Alternatively, the therapy can be pretreatment therapy.

As used herein, the term “combination therapy” refers to therapy in which the HDAC inhibitor and roscovitine are administered, if not simultaneously, then sequentially within a time frame that they both are available to act therapeutically within the same time frame.

As used herein, the term “pretreatment therapy” or “pretreated” means a regimen in which one agent is administered prior to, either separately or sequentially, the second agent. Preferably, the second agent is administered at least 2 hours after the administration of the first agent. More preferably, the second agent is administered at least 4 hours, or more preferably at least 6 or 8 hours, after the administration of the first agent. Even more preferably, the second agent is administered at least 12 hours, or more preferably at least 18 or 24 hours, after the administration of the first agent.

Preferably, roscovitine and the HDAC inhibitor interact in a synergistic manner. As used herein, the term “synergistic” means that roscovitine and the HDAC inhibitor produce a greater effect when used in combination than would be expected from adding the individual effects of the two components. Advantageously, a synergistic interaction may allow for lower doses of each component to be administered to a patient, thereby decreasing the toxicity of chemotherapy, whilst producing and/or maintaining the same therapeutic effect. Thus, in a particularly preferred embodiment, each component can be administered in a sub-therapeutic amount.

In another preferred embodiment, the roscovitine and the HDAC inhibitor interact in a manner so as to alleviate or eliminate adverse side effects associated with use of the individual components in monotherapy, or associated with their use in known combinations.

For all of the above embodiments, preferably the HDAC inhibitor is selected from sodium butyrate, or a prodrug thereof, suberoylanilide hydroxamic acid (SAHA) and trichostatin A (TSA).

### Proliferative Disorder

The term “proliferative disorder” is used herein in a broad sense to include any disorder that requires control of the cell cycle, for example cardiovascular disorders such as restenosis and cardiomyopathy, auto-immune disorders such as glomerulonephritis and rheumatoid arthritis, dermatological disorders such as psoriasis, anti-inflammatory, anti-fungal, antiparasitic disorders such as malaria, emphysema and alopecia. In these disorders, the compounds of the present invention may induce apoptosis or maintain stasis within the desired cells as required.

In respect of all of the above aspects and embodiments, preferably the proliferative disorder is cancer.

### Non-Small Cell Lung Cancer

In one particularly preferred embodiment of the invention, the proliferative disorder is lung cancer, more preferably, non-small cell lung cancer (NSCLC).

Lung cancers (bronchogenic carcinomas) may be divided into two broad categories namely, small cell lung cancer (SCLC) and non-small cell lung cancer (NSCLC). The distinction between these two types of cancer is based on the appearance of the tumour cells when viewed under a microscope.

SCLC accounts for 20% of lung cancers diagnosed and it is characterised by small cells which are mostly filled with the nucleus (hence the name). It is sometimes also referred to as “oat cell” cancer. SCLC is the most aggressive type of cancer, which metastasizes rapidly to other parts of the body. Diagnosis with SCLC often occurs only after the cancer has spread throughout the body. In general, SCLC is almost always caused as a result of smoking.

NSCLC can be subdivided into a group of related lung cancers which include epidermoid or squamous cell carcinoma, adenocarcinoma and large cell carcinoma.

Squamous cell lung cancer accounts for approximately 30% of all lung cancer cases and develops from reserve cells (which have the role of replacing damaged epithelium cells) in the lining of the lungs and bronchi. As a result, the cancer often initially develops in the centre of the chest. Squamous cell lung cancers are frequently slow growing and can take several years to progress from a confined tumour into invasive cancer. In 10-20% of cases, the cancer cavitates within the lungs. On metastasis, it often spreads to the bone, liver, adrenal glands, small intestine and brain.

Adenocarcinoma is the most common form of lung cancer making up 30-40% of all lung cancer cases. Adenocarcinoma develops in the outer part of the lung and develops from mucus-producing cells. The course of this cancer varies widely but often progresses slowly and the patient will present with few or no symptoms. In some cases, however, it can be extremely aggressive and rapidly fatal. In 50% of cases when it metastasises, it spreads only to the brain. Other locations to which adrenocarcinoma spreads include the liver, the adrenal glands, and bone.

The incidence of large cell carcinoma occurs less frequently than that of either adenocarcinoma or squamous cell carcinoma and accounts for 10-20% of lung cancer cases. The cancer is composed of large-sized cells that are anaplastic in nature and often arise in the bronchi. Large cell carcinoma develops on the periphery of the lungs and can spread to the plura.

Currently, lung cancer may be treated by surgery, radiation therapy or chemotherapy. Chemotherapy may be administered either alone or in combination with the other treatment options. Common NSCLC drugs and regimens include camptosar (irinotecan; CPT-11), camptothecin, carboplatin (paraplatin), cisplatin (platinol), epirubicin, gemcitabine, navelbine (vinorelbine), oxaliplatin, taxol (paclitaxel) and taxotere (docetaxol) (NSCLC Treatment—Chemotherapy, Lung Cancer Online).

However, chemotherapy is not curative. Other disadvantages of this treatment include toxicity, bystander damage to normal tissues and drug resistance (W. Wang et al, Cancer Sci., 2005, 96(10), 706). Furthermore, studies have shown that there is little survival benefit with some of the known treatments, such as vinorelbine (M. A. Socinski et al, Clin. Adv. Hematol. Oncol., 2003, 1(1), 33). Even a novel active such a troxacitabine has been shown to have little activity in NSCLC in 10 mg/m² doses administered intravenously over 30 minutes every three weeks (S. F. Dent et al, Lung, 2005, 183(4), 265).

The combination of gemcitabine/cisplatin has become widely used in Europe for the treatment of NSCLC. Cisplatin, however, is acknowledged to have certain disadvantages in that significant non-hematological toxicity (ototoxicity and nephroxicity) occurs in patients, along with emesis (P. Zatloukal et al, Lung Cancer, 2002, 38, S33).

As the outcome for a patient diagnosed with lung cancer is poor—the ten year survival rate for all treated cases is only approximately 8%—there exists a continuing need to develop effective treatments.

### Pharmaceutical Compositions

In a particularly preferred embodiment, the pharmaceutical product of the invention is in the form of a pharmaceutical composition comprising a pharmaceutically acceptable carrier, diluent or excipient.

Even though the compounds of the present invention (including their pharmaceutically acceptable salts, esters and pharmaceutically acceptable solvates) can be administered alone, they will generally be administered in admixture with a pharmaceutical carrier, excipient or diluent, particularly for human therapy. The pharmaceutical compositions may be for human or animal usage in human and veterinary medicine.

Examples of such suitable excipients for the various different forms of pharmaceutical compositions described herein may be found in the “Handbook of Pharmaceutical Excipients”, 2^{nd} Edition, (1994), Edited by A Wade and P J Weller.

Acceptable carriers or diluents for therapeutic use are well known in the pharmaceutical art, and are described, for example, in Remington's Pharmaceutical Sciences, Mack Publishing Co. (A. R. Gennaro edit. 1985).

Examples of suitable carriers include lactose, starch, glucose, methyl cellulose, magnesium stearate, mannitol, sorbitol and the like. Examples of suitable diluents include ethanol, glycerol and water.

The choice of pharmaceutical carrier, excipient or diluent can be selected with regard to the intended route of administration and standard pharmaceutical practice. The pharmaceutical compositions may comprise as, or in addition to, the carrier, excipient or diluent any suitable binder(s), lubricant(s), suspending agent(s), coating agent(s), solubilising agent(s).

Examples of suitable binders include starch, gelatin, natural sugars such as glucose, anhydrous lactose, free-flow lactose, beta-lactose, corn sweeteners, natural and synthetic gums, such as acacia, tragacanth or sodium alginate, carboxymethyl cellulose and polyethylene glycol.

Examples of suitable lubricants include sodium oleate, sodium stearate, magnesium stearate, sodium benzoate, sodium acetate, sodium chloride and the like.

Preservatives, stabilizers, dyes and even flavoring agents may be provided in the pharmaceutical composition. Examples of preservatives include sodium benzoate, sorbic acid and esters of p-hydroxybenzoic acid. Antioxidants and suspending agents may be also used.

### Salts/Esters

The agents of the present invention can be present as salts or esters, in particular pharmaceutically acceptable salts or esters.

Pharmaceutically acceptable salts of the agents of the invention include suitable acid addition or base salts thereof. A review of suitable pharmaceutical salts may be found in Berge et al, J Pharm Sci, 66, 1-19 (1977). Salts are formed, for example with strong inorganic acids such as mineral acids, e.g. sulphuric acid, phosphoric acid or hydrohalic acids; with strong organic carboxylic acids, such as alkanecarboxylic acids of 1 to 4 carbon atoms which are unsubstituted or substituted (e.g., by halogen), such as acetic acid; with saturated or unsaturated dicarboxylic acids, for example oxalic, malonic, succinic, maleic, fumaric, phthalic or tetraphthalic; with hydroxycarboxylic acids, for example ascorbic, glycolic, lactic, malic, tartaric or citric acid; with aminoacids, for example aspartic or glutamic acid; with benzoic acid; or with organic sulfonic acids, such as (C1-C4)-alkyl- or aryl-sulfonic acids which are unsubstituted or substituted (for example, by a halogen) such as methane- or p-toluene sulfonic acid.

Esters are formed either using organic acids or alcohols/hydroxides, depending on the functional group being esterified. Organic acids include carboxylic acids, such as alkanecarboxylic acids of 1 to 12 carbon atoms which are unsubstituted or substituted (e.g., by halogen), such as acetic acid; with saturated or unsaturated dicarboxylic acid, for example oxalic, malonic, succinic, maleic, fumaric, phthalic or tetraphthalic; with hydroxycarboxylic acids, for example ascorbic, glycolic, lactic, malic, tartaric or citric acid; with aminoacids, for example aspartic or glutamic acid; with benzoic acid; or with organic sulfonic acids, such as (C1-C4)-alkyl- or aryl-sulfonic acids which are unsubstituted or substituted (for example, by a halogen) such as methane- or p-toluene sulfonic acid. Suitable hydroxides include inorganic hydroxides, such as sodium hydroxide, potassium hydroxide, calcium hydroxide, aluminium hydroxide. Alcohols include alkanealcohols of 1-12 carbon atoms which may be unsubstituted or substituted, e.g. by a halogen).

### Enantiomers/Tautomers

The invention also includes where appropriate all enantiomers and tautomers of the agents. The man skilled in the art will recognise compounds that possess optical properties (one or more chiral carbon atoms) or tautomeric characteristics. The corresponding enantiomers and/or tautomers may be isolated/prepared by methods known in the art.

### Stereo and Geometric Isomers

Some of the agents of the invention may exist as stereoisomers and/or geometric isomers—e.g. they may possess one or more asymmetric and/or geometric centres and so may exist in two or more stereoisomeric and/or geometric forms. The present invention contemplates the use of all the individual stereoisomers and geometric isomers of those inhibitor agents, and mixtures thereof. The terms used in the claims encompass these forms, provided said forms retain the appropriate functional activity (though not necessarily to the same degree).

The present invention also includes all suitable isotopic variations of the agent or pharmaceutically acceptable salts thereof. An isotopic variation of an agent of the present invention or a pharmaceutically acceptable salt thereof is defined as one in which at least one atom is replaced by an atom having the same atomic number but an atomic mass different from the atomic mass usually found in nature. Examples of isotopes that can be incorporated into the agent and pharmaceutically acceptable salts thereof include isotopes of hydrogen, carbon, nitrogen, oxygen, phosphorus, sulphur, fluorine and chlorine such as 2H, 3H, 13C, 14C, 15N, 17O, 18O, 31P, 32P, 35S, 18F and 36C1, respectively. Certain isotopic variations of the agent and pharmaceutically acceptable salts thereof, for example, those in which a radioactive isotope such as 3H or 14C is incorporated, are useful in drug and/or substrate tissue distribution studies. Tritiated, i.e., 3H, and carbon-14, i.e., 14C, isotopes are particularly preferred for their ease of preparation and detectability. Further, substitution with isotopes such as deuterium, i.e., 2H, may afford certain therapeutic advantages resulting from greater metabolic stability, for example, increased in vivo half-life or reduced dosage requirements and hence may be preferred in some circumstances. Isotopic variations of the agent of the present invention and pharmaceutically acceptable salts thereof of this invention can generally be prepared by conventional procedures using appropriate isotopic variations of suitable reagents.

### Solvates

The present invention also includes solvate forms of the agents of the present invention. The terms used in the claims encompass these forms.

### Polymorphs

The invention furthermore relates to agents of the present invention in their various crystalline forms, polymorphic forms and (an)hydrous forms. It is well established within the pharmaceutical industry that chemical compounds may be isolated in any of such forms by slightly varying the method of purification and or isolation form the solvents used in the synthetic preparation of such compounds.

### Prodrugs

The invention further includes agents of the present invention in prodrug form. Such prodrugs are generally compounds wherein one or more appropriate groups have been modified such that the modification may be reversed upon administration to a human or mammalian subject. Such reversion is usually performed by an enzyme naturally present in such subject, though it is possible for a second agent to be administered together with such a prodrug in order to perform the reversion in vivo. Examples of such modifications include ester (for example, any of those described above), wherein the reversion may be carried out be an esterase etc. Other such systems will be well known to those skilled in the art.

### Administration

The pharmaceutical compositions of the present invention may be adapted for oral, rectal, vaginal, parenteral, intramuscular, intraperitoneal, intraarterial, intrathecal, intrabronchial, subcutaneous, intradermal, intravenous, nasal, buccal or sublingual routes of administration.

For oral administration, particular use is made of compressed tablets, pills, tablets, gellules, drops, and capsules. Preferably, these compositions contain from 1 to 2000 mg and more preferably from 50-1000 mg, of active ingredient per dose.

Other forms of administration comprise solutions or emulsions which may be injected intravenously, intraarterially, intrathecally, subcutaneously, intradermally, intraperitoneally or intramuscularly, and which are prepared from sterile or sterilisable solutions. The pharmaceutical compositions of the present invention may also be in form of suppositories, pessaries, suspensions, emulsions, lotions, ointments, creams, gels, sprays, solutions or dusting powders.

An alternative means of transdermal administration is by use of a skin patch. For example, the active ingredient can be incorporated into a cream consisting of an aqueous emulsion of polyethylene glycols or liquid paraffin. The active ingredient can also be incorporated, at a concentration of between 1 and 10% by weight, into an ointment consisting of a white wax or white soft paraffin base together with such stabilisers and preservatives as may be required.

Injectable forms may contain between 10-1000 mg, preferably between 10-500 mg, of active ingredient per dose.

Compositions may be formulated in unit dosage form, i.e., in the form of discrete portions containing a unit dose, or a multiple or sub-unit of a unit dose.

In a particularly preferred embodiment, the combination or pharmaceutical composition of the invention is administered intravenously.

### Dosage

A person of ordinary skill in the art can easily determine an appropriate dose of one of the instant compositions to administer to a subject without undue experimentation. Typically, a physician will determine the actual dosage which will be most suitable for an individual patient and it will depend on a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy. The dosages disclosed herein are exemplary of the average case. There can of course be individual instances where higher or lower dosage ranges are merited, and such are within the scope of this invention.

Depending upon the need, the agent may be administered at a dose of from 0.1 to 30 mg/kg body weight, such as from 2 to 20 mg/kg, more preferably from 0.1 to 1 mg/kg body weight.

By way of guidance, the HDAC inhibitor is typically administered in accordance with a physician's direction at dosages described in the relevant references discussed above. Pivanex is typically administered at about 2.34 g/m² per day. Pivanex is preferably administered intravenously. Suberoylanilide hydroxamic acid (SAHA) is typically administered from about 100-600 mg per day. Suberoylanilide hydroxamic acid (SAHA) is preferably administered orally. The total daily dose of HDAC inhibitor can be administered as a single dose or divided into separate dosages preferably administered two, three or four time a day.

Roscovitine is typically administered from about 0.05 to about 5 g/day, preferably from about 0.4 to about 3 g/day. Roscovitine is preferably administered orally in tablets or capsules. The total daily dose of roscovitine can be administered as a single dose or divided into separate dosages preferably administered two, three or four time a day.

In one preferred embodiment, roscovitine is administered as an orally or intravenously at a dosage of from 0.4 to 3 g/day and the HDAC inhibitor is administered in the manner deemed most suitable at an appropriate dosage as discussed above. Preferably, the HDAC inhibitor is administered at least 2 hours before the administration of roscovitine. More preferably, the HDAC inhibitor is administered at least 4 hours, or more preferably at least 6 or 8 hours, before the administration of roscovitine. Even more preferably, the HDAC inhibitor is administered at least 12 hours, or more preferably at least 18 or 24 hours, before the administration of roscovitine.

In another preferred embodiment, the HDAC inhibitor is administered at least 2 hours after the administration of roscovitine. More preferably, the HDAC inhibitor is administered at least 4 hours, or more preferably at least 6 or 8 hours, after the administration of roscovitine. Even more preferably, the HDAC inhibitor is administered at least 12 hours, or more preferably at least 18 or 24 hours, after the administration of roscovitine.

### Kit of Parts

A further aspect of the invention relates to a kit of parts comprising:
(i) roscovitine, or a pharmaceutically acceptable salt thereof, optionally admixed with a pharmaceutically acceptable diluent, excipient or carrier; and
(ii) a HDAC inhibitor selected from sodium butyrate, or a prodrug thereof, suberoylanilide hydroxamic acid (SAHA), sodium valproate and trichostatin A (TSA), optionally admixed with a pharmaceutically acceptable diluent, excipient or carrier.

Preferably, the roscovitine and the HDAC inhibitor are each in unit dosage form. Preferably, the kit of parts contains a plurality of unit dosage forms of each component, i.e. of components (i) and (ii) above.

Optionally, the kit of parts may further comprise a means for facilitating compliance with a particular dosing regimen, for example, instructions indicating when, how, and how frequently the unit dosage forms of each component should be taken.

The present invention is further described by way of example, and with reference to the following figures, wherein:

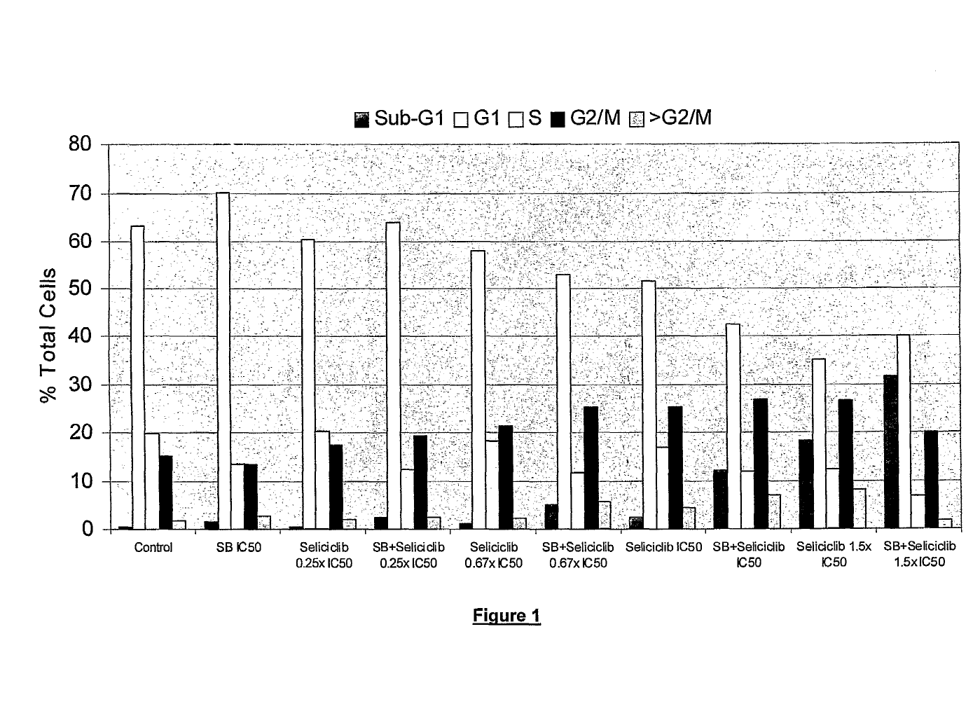

FIG. 1 shows that concomitant treatment of seliciclib and sodium butyrate leads to a synergistic increase in apoptosis in A549 cells, as determined by an increase in sub-G1 cell fragments, following 72 hours treatment.

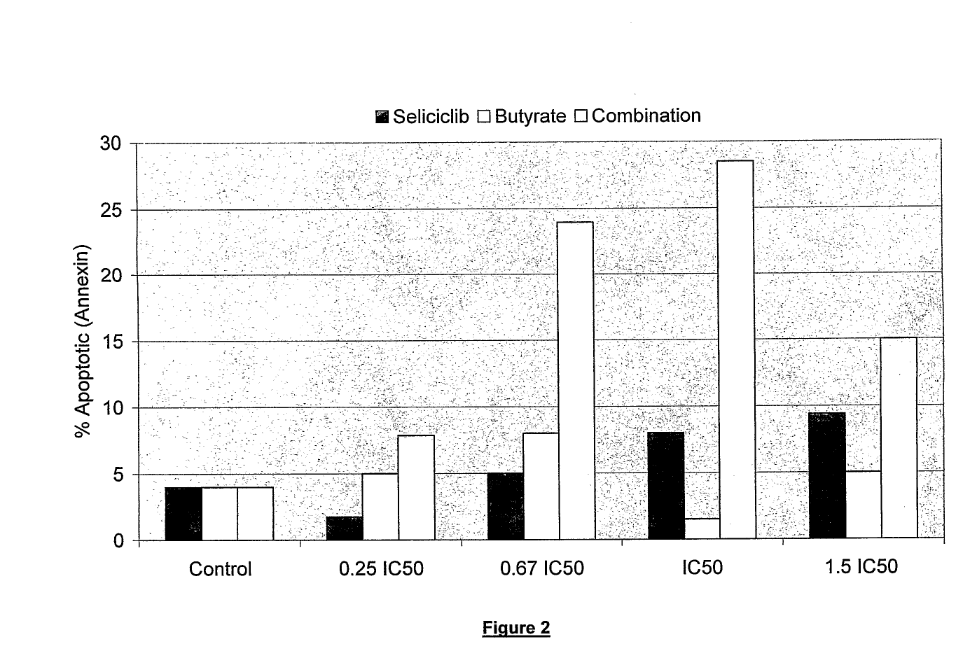

FIG. 2 shows that concomitant treatment of seliciclib and sodium butyrate in H460 cells leads to a synergistic increase in apoptosis following 72 hours treatment, as determined by annexin V staining.

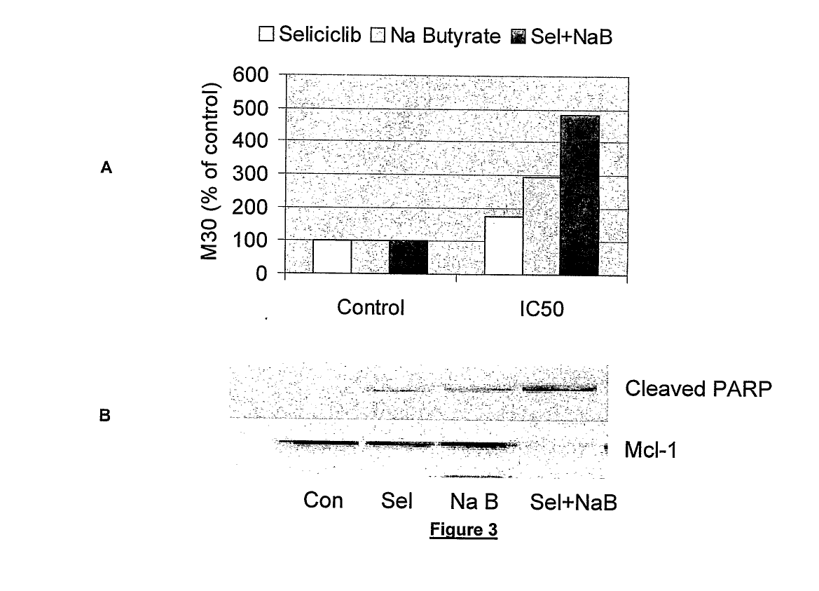

FIG. 3 shows that in respect of the seliciclib/sodium butyrate combination there is an increase in apoptosis at the IC50 concentration, as determined by caspase cleavage of cytokeratin 18 (M30 ELISA) and PARP cleavage. In addition, FIG. 3 also shows a synergistic decrease in Mcl1 levels which probably relates to the loss of this anti-apoptotic protein, pushing the cells into apoptosis.

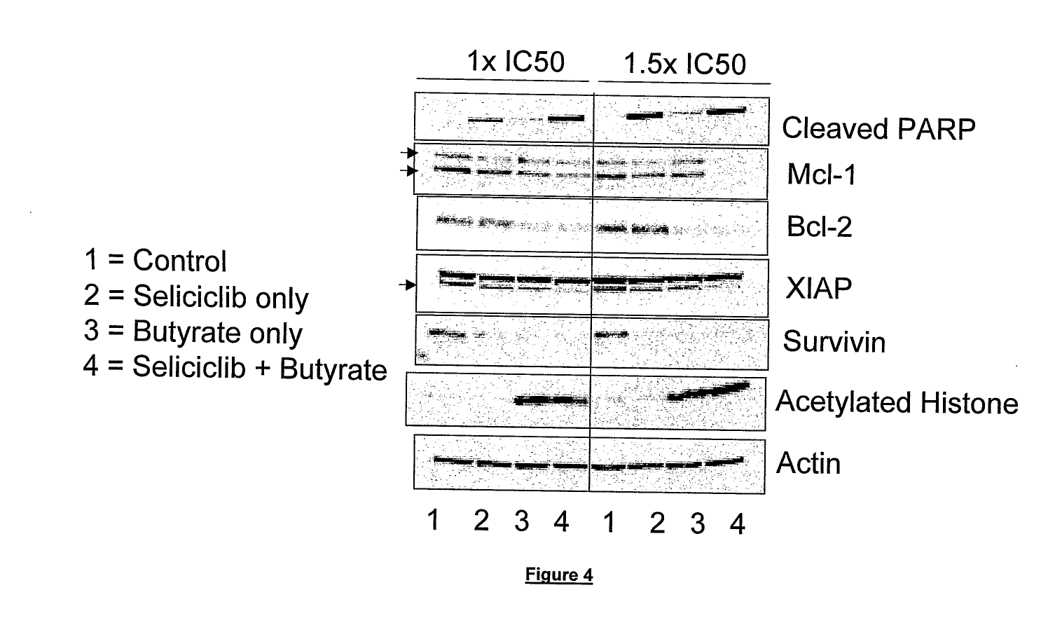

FIG. 4 shows the molecular pathways of apoptosis with regard to the seliciclib/sodium butyrate combination in more detail. Loss of Mcl1 and Bcl2 (both anti-apoptotic proteins) pushes the cells towards apoptosis. Both XIAP and survivin are inhibitors of the apoptotic process therefore the loss of these proteins again push the cells towards apoptosis. As the decreases in XIAP and Mcl1 are synergistic, this effect may explain the synergistic induction of apoptosis as shown by the appearance of PARP. The Histone Western blot shows that the HDAC inhibitor increases the amount of acetylated histone (since deacetylation is inhibited).

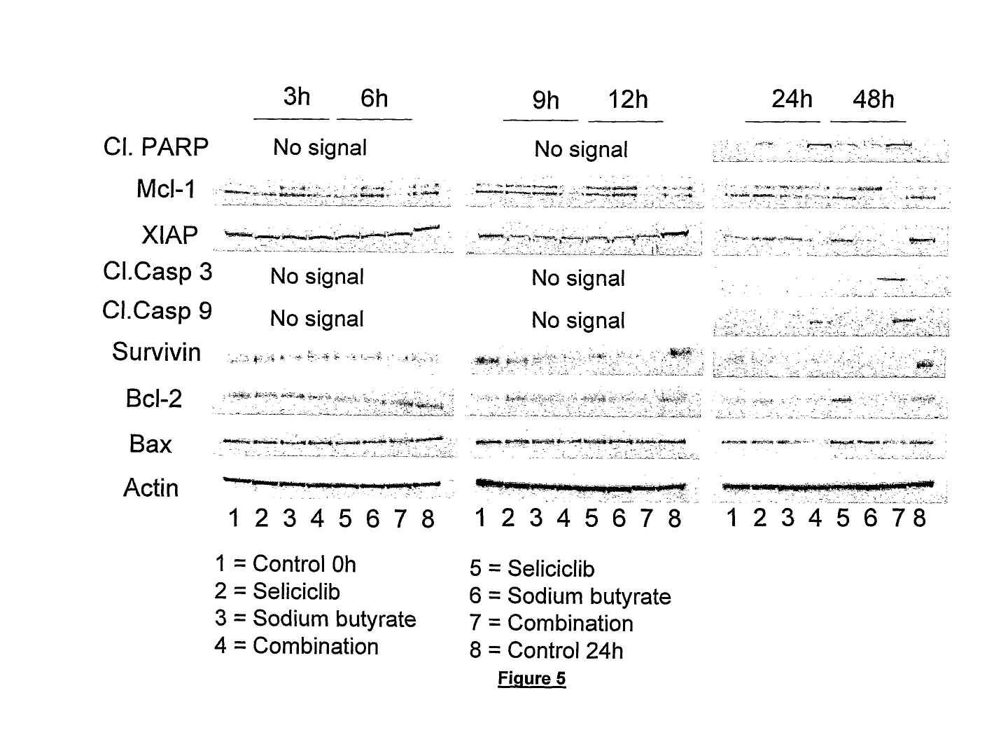

FIG. 5 shows the time course of cellular events at the IC50 in respect of the seliciclib/sodium butyrate combination. At later time points, the synergistic activation of caspases 3 and 9 (indicating apoptosis is induced) can now be seen.

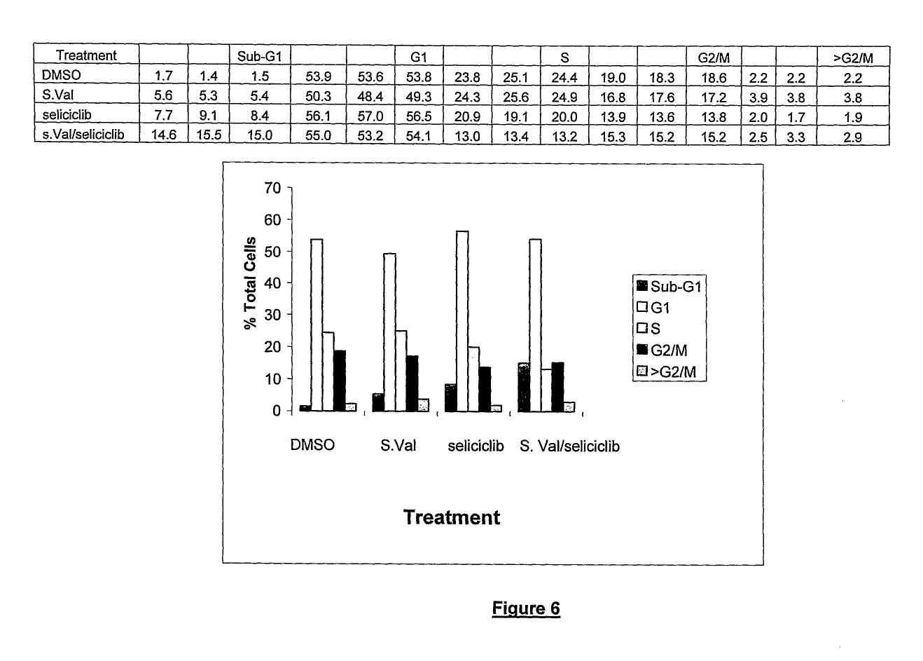

FIG. 6 shows the cell cycle distribution after treatment with DMSO (control), sodium valproate, seliciclib and sodium valproate/seliciclib in combination. H460 cells were treated with the indicated drug(s) for the times shown prior to PI analysis on the flow cytometer. The results are the average of two duplicate samples.

### EXAMPLES

### Methods

### R-Roscovitine

R-Roscovitine was prepared in accordance with the method disclosed in EP0874847B (CNRS).

### HDAC Inhibitors

Sodium butyrate and sodium valproate were obtained from Sigma; TSA was obtained from AG Scientific, Inc.; SAHA was obtained from Toronto Research Chemicals, Inc.

### Cell Culture

Experiments were carried out in 96-well plates and the cell lines seeded at a density of 2000/well for A549 and 3000/well for H460. The IC50 values after 24 h treatment and 72 h treatment were determined for sodium butyrate in each cell line and SAHA, sodium valproate and TSA in H460 cells, using the Alamar blue assay. Each HDAC inhibitor was then tested in combination with seliciclib using three different treatment regimes: concomitant, seliciclib pre-treatment followed by HDAC inhibitor and HDAC inhibitor pretreatment followed by seliciclib.

### Calcusyn Drug Combination Protocol

For the concomitant treatment, 1.5-fold serial dilutions of seliciclib, HDAC inhibitor, or both drugs simultaneously were added to cells 24 h after plating, and left for 72 h at 37° C. The drug concentrations selected were chosen to span the IC₅₀ values for the drugs tested. In the pre-treatment regimes, the first drug was added 2 h after cells were plated, and left for 24 h. Medium was aspirated and replaced with fresh medium containing the second drug, and incubated for 72 h. The two controls for each sequential treatment involved substituting one of the drug treatments with medium. After drug treatment, the cell number in each well was then estimated by incubating the cells for 1 h in medium containing 10% alamar blue (Roche, Lewes, East Sussex, U.K.) and reading the absorbance at 544-595 nm. Drug interactions were analysed using the commercial software package Calcusyn, which is based on the median effect model of Chou and Talalay (Chou, T. C. & Talalay, P. (1984) Adv. Enzyme Regul. 22, 27-55. Quantatative analysis of dose-effect relationships: the combined effects of multiple drugs or enzyme inhibitors). A Combination Index (C.I.) of 1 indicated an additive drug interaction, whereas a C.I. greater than 1 was antagonistic and a score lower than 1 was synergistic.

### Western Blotting Analysis

Protein lysates were generated from 10 cm plates that were seeded at approximately 5×10⁵ cells/well, in medium containing 10% FCS. Cells were incubated with HDAC inhibitor and/or seliciclib at the indicated concentrations and times prior to harvest. After incubation, the supernatants were removed and centrifuged at 200 rpm for 5 min to pellet any floating cells. Cells on the plates were washed once with ice-cold buffer A (50 mM HEPES, pH 7.0, containing 20 mM NaCl), then each plate was scraped into 0.15 ml buffer A containing 1 mM DTT, protease inhibitors (diluted 1:1000 into buffer A) and phosphatase inhibitors (10 mM Sodium pyrophosphate, 10 mM Sodium Fluoride and 1 mM Sodium Orthovanadate). The supernatant cell pellets were resuspended with 50 μl buffer A containing DTT, protease and phosphatase inhibitors and pooled with the appropriate sample from the plates. Cells were lysed by sonication (2×3s bursts with probe sonicator), and the protein concentration of each tube determined using the BCA assay. Lysates (20-30 μg protein loaded/well) were resolved on Bis-Tris gels containing 12% acrylamide and transferred to nitrocellulose for analysis by western blotting. Membranes were blocked for 1 h at room temperature in PBS containing 0.02% (v/v) Tween 20 and 5% (w/v) fat-free dried milk. Antibody incubations were carried out overnight at 2-8° C. in PBS containing 0.02% (v/v) Tween 20 and 3% (w/v) dried milk. Nitrocellulose membranes were probed with the following antibodies:

| | | | Dilution |
|---|---|---|---|
| Antibody | Source | Target protein | used |
| Actin (A5441) | Sigma | β-Actin | 1:15000 |
| Cleaved PARP | BD Pharmingen | Cleaved PARP | 1:500 |
| Acetyl-Histone H4 | Upstate | Acetylated Histone H4 | 1:1000 |
| XIAP | Cell Signalling | XIAP | 1:1000 |
| Bcl-2 (clone100) | Upstate | Bcl-2 | 1:1000 |
| Mcl-1 (S-19) | Santa Cruz | Mcl-1 | 1:1000 |
| Bax (06-499) | Upstate | Bax | 1:1000 |
| Survivin | AbCam | Survivin | 1:500 |
| Cleaved Caspase 3 | Cell Signalling | Cleaved Caspase 3 | 1:1000 |
| Cleaved Caspase 9 | Cell Signalling | Cleaved Caspase 9 | 1:1000 |

### Flow Cytometry: Seliciclib/Sodium Butyrate Combination

H460 cells were seeded in 10 cm plates at approximately 3×10⁵ cells/plate and left to settle overnight. Next day, seliciclib, sodium butyrate or both drugs were added at the indicated concentrations. After either 24 h or 72 h treatment, cells were harvested by trypsinisation. Cell cycle analysis by propidium iodide (PI) staining involved fixing the cells overnight in 70% (v/v) ethanol at −20° C. prior to analysis on the flow cytometer. Annexin V staining was performed as indicated in manufacturers instructions, on live, non-fixed cells.

### Flow Cytometry: Seliciclib/Sodium Valproate Combination

H460 cells were seeded onto 10 cm plates at approximately 0.5×10⁶ cells/plate and allowed to settle for 24 h. Cells were treated with sodium valproate for 24 hours followed by seliciclib for a further 24 hours. The concentrations of compound used were equivalent to 1×IC₅₀ Single agent control treatments were also carried out. These involved treating the cells with sodium valproate for 24 hours followed by drug-free medium for a further 24 hours, or drug-free medium for 24 hours followed by seliciclib for a further 24 hours. All cells were harvested by collecting the media prior to media changes, as well as at 48 hours. Adherent cells were harvested by trypsinisation, pooled with the cells in suspension, washed twice in PBS and fixed by resuspending in 1 ml ice-cold 70% ethanol. Standard cell cycle analysis by propidium iodide staining was carried out on the flow cytometer. Results are the average of duplicate samples.

### M30/TPS Analysis of Time-Gap Experiment

A549 cells were seeded in 96 well plates and left to settle overnight. Cells were treated with seliciclib, sodium butyrate or the combination at the indicated concentrations. After 72 h treatment, medium was harvested, retained and stored at −20° C. Samples were analysed in the M30 ELISA as described in the manufacturers instructions.

### Results

### Seliciclib and HDAC Inhibitors in Combination in NSCLC Cell Lines.

Seliciclib was tested in combination with the indicated HDAC inhibitors in H460 and A549 cell lines, using three different treatment regimes. The Combination Index values from each drug treatment are shown for ED50, ED75 and ED90 values (the point on the curve where 50%, 75% and 90% of the cells have been killed). Data are the average of at least three independent experiments (Table 1).

TABLE 1
Data for the effect of seliciclib and the HDAC inhibitors
on the A549 cell lines are shown in parentheses;

| | | | HDAC | |
|---|---|---|---|---|
| HDAC | | Seliciclib | inhibitor | Concomitant |
| Inhibitor | Effect | pretreatment | pretreatment | treatment |
| Butyrate | ED50 | 0.88 (1.08) | 0.74 (0.95) | 0.82 (1.21) |
| | ED75 | 0.68 (0.88) | 0.53 (0.71) | 0.59 (0.87) |
| | ED90 | 0.57 (0.75) | 0.40 (0.60) | 0.44 (0.64) |
| SAHA | ED50 | 1.15 | 0.92 | 1.17 |
| | ED75 | 1.04 | 0.81 | 1.09 |
| | ED90 | 0.97 | 0.71 | 1.02 |
| TSA | ED50 | 0.92 | 0.91 | 1.08 |
| | ED75 | 0.73 | 0.80 | 0.91 |
| | ED90 | 0.59 | 0.71 | 0.78 |
| Sodium | ED90 | 0.81 | 0.84 | 1.07 |
| valproate | | | | |

These results demonstrate that seliciclib and butyrate are synergistic in H460 and A549 cells, with all three treatment regimes tested. Seliciclib and trichostatin A are moderately synergistic in H460 cells, with all three treatment regimes tested. Seliciclib and SAHA are moderately synergistic in H460 cells when SAHA pre-treatment is followed by seliciclib, and additive with the other two treatment regimes. Synergy was observed for sequential treatment with seliciclib and sodium valproate, irrespective of the order of administration. Therefore, if cells are pretreated with the HDAC inhibitor, seliciclib is synergistic when used in combination with all four HDAC inhibitors tested, demonstrating that combining seliciclib with a HDAC inhibitor is a good concept for treating NSCLC cell lines.

### Flow Cytometry Studies

### Seliciclib and Butyrate Induce a Synergistic Increase in Sub-G1 A549 Cells.

A549 cells were incubated with IC50 butyrate, 0.25-1.5×IC₅₀ seliciclib, or 0.25-1.5×IC50 seliciclib in the presence of IC50 butyrate for 72 h. Cells were then harvested, stained with propidium iodide and their DNA content analysed by flow cytometry. Data are representative of two independent experiments (FIG. 1).

Butyrate alone induced a small increase in sub-G1 cells (<2n DNA), which are dead or undergoing apoptosis. Seliciclib treatment induced a dose-dependent increase in sub-G1 cells, which was synergistically enhanced by inclusion of butyrate. These data indicate that seliciclib and butyrate induce a synergistic increase in cells that are dead or dying.

Seliciclib and Butyrate Induce a Synergistic Increase in Apoptotic H460 Cells.

H460 cells were incubated with 0.25-1.5×IC50 butyrate, 0.25-1.5×IC50 seliciclib, or 0.25-1.5×IC50 seliciclib and butyrate for 72 h. Cells were then harvested, stained with annexin V and analysed on the flow cytometer. Data are representative of two independent experiments (FIG. 2).

Annexin V labels live cells that are undergoing apoptosis. At 0.67× and 1×IC50 concentrations, butyrate and seliciclib induced a much larger annexin V signal than the two single drug treatments combined, indicating a synergistic increase in apoptotic cells. The highest concentration of butyrate and seliciclib (1.5×IC50) appears to contain fewer cells undergoing apoptosis than those treated with 0.67× or 1×IC50, the reason for this is not clear at present.

### Seliciclib and Butyrate Synergistically Induce Apoptosis in A549 Cells.

A549 cells were treated with DMSO (control) or with IC50 concentrations of seliciclib, sodium butyrate, or seliciclib and butyrate for 72 h, as indicated. Cell culture supernatants were harvested and tested in the M30 apoptosense ELISA, and the cells harvested and analysed for cleaved PARP and Mcl-1 by western blotting. Data are representative of two independent experiments (FIG. 3).

The results show that seliciclib and butyrate together give a larger M30 signal than the two individual drug treatments combined, indicating that they synergistic increase apoptosis. This data is supported by the fact that seliciclib and butyrate give a larger cleaved PARP signal than seliciclib or butyrate alone, which is indicative of an additive/synergistic increase in apoptosis. Seliciclib and butyrate also decreased the level of the anti-apoptotic protein Mcl-1 in A549 cells, whereas the single drug treatments had no significant effect on this protein. This could play a role in promoting the increased apoptosis that was detected.

### Seliciclib and Butyrate Regulate Several Apoptotic Proteins in a Dose-Dependent Manner.

H460 cells were treated with butyrate, seliciclib or seliciclib and butyrate at 1× or 1.5×IC50 concentrations for 24 h. Cells were harvested and the resulting cell lysates analysed by western blotting with the indicated antibodies. Data are representative of two independent experiments (FIG. 4).

The data indicate that by 24 h treatment, seliciclib and butyrate synergistically decrease the protein levels of the anti-apoptotic protein Mcl-1 and the caspase inhibitor XIAP. Butyrate treatment reduces the levels of the anti-apoptotic protein Bcl-2 and the caspase inhibitor survivin. Together, these changes will provide a strong pro-apoptotic signal. Indeed, there is a synergistic increase in cleaved PARP at 1×IC50 concentrations, although the increased cleaved PARP at 1.5×IC50 only appears to be an additive effect at best, which is in agreement with the annexin V data generated at 1.5×IC50 in FIG. 2.

### Seliciclib and Butyrate Regulate Several Apoptotic Proteins in a Time-Dependent Manner.

H460 cells were treated with 1×IC50 butyrate, seliciclib or seliciclib and butyrate for the indicated times. Cells were harvested and the resulting cell lysates analysed by western blotting with the indicated antibodies. Data are representative of two independent experiments (FIG. 5).

The results confirm the observations in FIG. 4, since butyrate reduces the levels of survivin and Bcl-2, and seliciclib and butyrate synergistically reduce Mcl-1 and XIAP levels. These data also demonstrate that the drug combination induces a synergistic increase in the active forms of caspase 3 and 9, which appear at around the same time as the increase in cleaved PARP. Interestingly, the increased apoptosis (as measured by the appearance of cleaved PARP) does not occur until after the decreases in Mcl-1, Bcl-2, survivin and XIAP, indicating that any or all of these changes may be required to promote the apoptotic effect in this cell line.

### Seliciclib/Sodium Valproate Combination

Flow cytometry analysis of the sodium valproate/seliciclib combination was carried out as described above. The sodium valproate pre-treatment schedule was selected as this was synergistic by Calcusyn. In H460 cells, treatment with seliciclib induced a small increase in the percentage of cells in sub-G1 (apoptotic cells), but otherwise had no significant impact on the overall cell cycle distribution of H460 cells at the concentrations used (FIG. 6).

Combining sodium valproate with seliciclib caused a significant increase in the proportion of sub-G1 (apoptotic) cells that, compared to the sum of the single agent controls, is in agreement with the data obtained by Calcusyn analysis.

The data illustrated above provides evidence that seliciclib appears to be synergistic with the HDAC inhibitor, sodium valproate, in H460 cells in a schedule-independent manner.

Various modifications and variations of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in the relevant fields are intended to be covered by the present invention.

1. A combination comprising roscovitine, or a pharmaceutically acceptable salt thereof, and a HDAC inhibitor selected from sodium butyrate, or a prodrug thereof, suberoylanilide hydroxamic acid (SAHA), sodium valproate and trichostatin A (TSA).

2. A combination according to claim 1 wherein the HDAC inhibitor is sodium butyrate, or a prodrug thereof.

3. A combination according to claim 2 wherein the prodrug is pivaloyloxymethyl butyrate.

4. A combination according to claim 1 wherein the HDAC inhibitor is suberoylanilide hydroxamic acid (SAHA).

5. A combination according to claim 1 wherein the HDAC inhibitor is trichostatin A (TSA).

6. A combination according to claim 1 wherein the HDAC inhibitor is sodium valproate.

7. A combination according to any preceding claim wherein roscovitine is R-roscovitine.

8. A pharmaceutical composition comprising a combination according to any preceding claim and a pharmaceutically acceptable carrier, diluent or excipient.

9. Use of a combination according to any one of claims 1 to 7 in the preparation of a medicament for treating a proliferative disorder.

10. A pharmaceutical product comprising roscovitine, or a pharmaceutically acceptable salt thereof, and a HDAC inhibitor selected from sodium butyrate, or a prodrug thereof, suberoylanilide hydroxamic acid (SAHA), sodium valproate and trichostatin A (TSA), as a combined preparation for simultaneous, sequential or separate use in therapy.

11. A pharmaceutical product according to claim 10 wherein the HDAC inhibitor is sodium butyrate, or a prodrug thereof.

12. A pharmaceutical product according to claim 11 wherein the prodrug is pivaloyloxymethyl butyrate.

13. A pharmaceutical product according to claim 10 wherein the HDAC inhibitor is suberoylanilide hydroxamic acid (SAHA).

14. A pharmaceutical product according to claim 10 wherein the HDAC inhibitor is trichostatin A (TSA).

15. A pharmaceutical product according to claim 10 wherein the HDAC inhibitor is sodium valproate.

16. A pharmaceutical product according to any one of claims 10 to 15 wherein the roscovitine is R-roscovitine.

17. A pharmaceutical product according to any one of claims 10 to 16 in the form of a pharmaceutical composition comprising a pharmaceutically acceptable carrier, diluent or excipient.

18. A pharmaceutical product according to any one of claims 10 to 17 for use in the treatment of a proliferative disorder.

19. A pharmaceutical product according to claim 18 wherein the proliferative disorder is cancer.

20. A pharmaceutical product according to claim 19 wherein the cancer is non small cell lung cancer (NSCLC).

21. A method of treating a proliferative disorder, said method comprising simultaneously, sequentially or separately administering roscovitine, or a pharmaceutically acceptable salt thereof, and a HDAC inhibitor selected from sodium butyrate, or a prodrug thereof, suberoylanilide hydroxamic acid (SAHA), sodium valproate and trichostatin A (TSA).

22. A method according to claim 21 wherein the roscovitine is R-roscovitine.

23. A method according to claim 21 or claim 22 wherein the prodrug of sodium butyrate is pivaloyloxymethyl butyrate.

24. A method according to any one of claims 21 to 23 wherein roscovitine, or pharmaceutically acceptable salt thereof, and the HDAC inhibitor are each administered in a therapeutically effective amount with respect to the individual components.

25. A method according to any of claims 21 to 23 wherein roscovitine, or pharmaceutically acceptable salt thereof, and the HDAC inhibitor are each administered in a sub-therapeutic amount with respect to the individual components.

26. A method according to any one of claims 21 to 25 wherein the HDAC inhibitor and roscovitine, or pharmaceutically acceptable salt thereof, are administered simultaneously.

27. A method according to any one of claims 21 to 25 wherein the HDAC inhibitor and roscovitine, or pharmaceutically acceptable salt thereof, are administered sequentially or separately.

28. A method according to claim 27 wherein the HDAC inhibitor is administered sequentially or separately prior to roscovitine or pharmaceutically acceptable salt thereof.

29. A method according to claim 27 wherein roscovitine, or a pharmaceutically acceptable salt thereof, is administered sequentially or separately prior to the HDAC inhibitor.

30. A method according to any one of claims 21 to 29 wherein the proliferative disorder is cancer.

31. A method according to claim 30 wherein the cancer is non small cell lung cancer (NSCLC).

32. Use of roscovitine, or a pharmaceutically acceptable salt thereof, in the preparation of a medicament for the treatment of a proliferative disorder, wherein said treatment comprises simultaneously, sequentially or separately administering a HDAC inhibitor selected from sodium butyrate, or a prodrug thereof, suberoylanilide hydroxamic acid (SAHA), sodium valproate and trichostatin A (TSA) to a subject.

33. Use of a HDAC inhibitor selected from sodium butyrate, or a prodrug thereof, suberoylanilide hydroxamic acid (SAHA), sodium valproate and trichostatin A (TSA) in the preparation of a medicament for the treatment of a proliferative disorder, wherein said treatment comprises simultaneously, sequentially or separately administering to a subject roscovitine, or a pharmaceutically acceptable salt thereof.

34. Use of roscovitine, or a pharmaceutically acceptable salt thereof, and a HDAC inhibitor selected from sodium butyrate, or a prodrug thereof, suberoylanilide hydroxamic acid (SAHA), sodium valproate and trichostatin A (TSA), in the preparation of a medicament for treating a proliferative disorder.

35. Use of a HDAC inhibitor selected from sodium butyrate, or a prodrug thereof, suberoylanilide hydroxamic acid (SAHA) and trichostatin A (TSA), in the preparation of a medicament for the treatment of a proliferative disorder, wherein said medicament is for use in combination therapy with roscovitine, or a pharmaceutically acceptable salt thereof.

36. Use of roscovitine, or a pharmaceutically acceptable salt thereof, in the preparation of a medicament for the treatment of a proliferative disorder, wherein said medicament is for use in combination therapy with a HDAC inhibitor selected from sodium butyrate, or a prodrug thereof, suberoylanilide hydroxamic acid (SAHA), sodium valproate and trichostatin A (TSA).

37. Use of roscovitine, or a pharmaceutically acceptable salt thereof, in the preparation of a medicament for the treatment of a proliferative disorder, wherein said medicament is for use in pretreatment therapy with a HDAC inhibitor selected from sodium butyrate, or a prodrug thereof, suberoylanilide hydroxamic acid (SAHA), sodium valproate and trichostatin A (TSA).

38. Use according to any one of claims 32 to 37 wherein the roscovitine is R-roscovitine.

39. A use according to any one of claims 32 to 38 wherein the prodrug of sodium butyrate is pivaloyloxymethyl butyrate.

40. Use according to any one of claims 32 to 39 wherein the proliferative disorder is cancer.

41. Use according to claim 40 wherein the cancer is non small cell lung cancer (NSCLC).

42. Use of roscovitine, or a pharmaceutically acceptable salt thereof, and a HDAC inhibitor in the preparation of a medicament for treating non small cell lung cancer.

43. Use of roscovitine, or a pharmaceutically acceptable salt thereof, and a HDAC inhibitor selected from sodium butyrate, or a prodrug thereof, suberoylanilide hydroxamic acid (SAHA), sodium valproate and trichostatin A (TSA), in the preparation of a medicament for treating non small cell lung cancer.

44. A kit of parts comprising:
(i) roscovitine, or a pharmaceutically acceptable salt thereof, optionally admixed with a pharmaceutically acceptable diluent, excipient or carrier; and
(ii) a HDAC inhibitor selected from sodium butyrate, or a prodrug thereof, suberoylanilide hydroxamic acid (SAHA), sodium valproate and trichostatin A (TSA), optionally admixed with a pharmaceutically acceptable diluent, excipient or carrier.

45. A combination, pharmaceutical composition, pharmaceutical product, method, use or kit of parts substantially as described herein.
